(19) United States
(12) Patent Application Publication
(10) Pub. No.: US 2024/0261133 A1
(43) Pub. Date: Aug. 8, 2024

(54) CASE FOR THE STORAGE AND EASY WEARING OF CONDOMS AND SINGLE-USE PACKAGE FOR CONDOMS

(71) Applicants: Davide MARIANI, Brescia (IT); Fabio MASCIALINO, Miami, FL (US)

(72) Inventors: Valerio FAUSTI, Milano (IT); Davide MARIANI, Brescia (IT); Elia MILANI, Milano (IT)

(21) Appl. No.: 18/564,868

(22) PCT Filed: May 24, 2022
(86) PCT No.: PCT/IB2022/054844

§ 371 Date: Nov. 28, 2023

(30) Foreign Application Priority Data
May 28, 2021 (IT) 102021000014018
May 28, 2021 (IT) 102021000014024

(51) Int. Cl. A61F 6/00; A61F 6/04

(52) U.S. Cl. CPC A61F 6/005; A61F 6/04

(57) ABSTRACT

A case for the storage and easy wearing of condoms emprises-includes a box-shaped casing defining a containment chamber configured to accommodate a plurality of condoms arranged in respective flattened single-use packages arranged to form a stack. A pusher operates in the containment chamber so that a terminal single-use package of said stack is held against a bottom wall of the chamber itself. An extractor movable between a waiting position in which it engages the terminal single-use package inside the containment chamber and an extracted position in which the terminal single-use package is extracted through an outlet slot arranged in proximity to the bottom wall to expose the respective condom outside the box-shaped casing, in a use condition suitable for wearing.

The present invention relates to a case for the storage and easy wearing of condoms, more specifically male condoms. The invention also relates to a single-use package for condoms, which can also be conveniently used in combination with the above-mentioned case.

As known, the use of condoms is an important aid not only to birth control, but also and above all to combating the spread of sexually transmitted diseases.

Condoms are typically packaged individually in single-use packages, consisting of an envelope-shaped casing made of a layered and heat-sealed material. Such packages must necessarily have a good mechanical strength to protect the content from possible damage due to abrasion or accidental contact with objects when stored, for example, in a pocket, bag or wallet.

This requirement makes it rather difficult to open the package, as a force sufficient to break the casing is needed while taking due care not to damage the condom during opening. The operations required to extract the condom out of the casing and wear it correctly also require a certain amount of care and skill. These complications tend to discourage the use of condoms, as they significantly interfere with the sensuality, intimacy and spirituality of the bond between the partners approaching the sexual act. In fact, it has been proven that among many individuals, especially men, a strong psychological resistance to condom use persists, and one of the causes is anxiety caused by the fear of losing an erection in the act of wearing a condom.

The object of the present invention is essentially to eliminate or at least limit the problems encountered in the prior art, by proposing a system that offers the possibility of safely and discreetly storing one or more condoms in a small space, making them readily available to be worn with simple and immediate gestures.

In particular, it was found that by arranging condoms in single-use packages enclosed in a box-shaped case made of a rigid material, the materials and mechanical strength of the individual single-use packages can be lightened, so that opening the packages is not too difficult. In addition, by providing appropriate thrust and abutment shoulders in the single-use package, a way was found to automate the extraction and opening of the individual packages so that, with an extremely simple operation, the individual condom is immediately exposed in a ready-to-use condition, wherein it is properly supported to facilitate wearing it on the erect penis.

More particularly, a case for the storage and easy wearing of condoms is an object of the present invention, comprising: a box-shaped casing defining a containment chamber configured to accommodate a plurality of condoms each arranged in a single-use package of substantially flattened shape in a respective lying plane, the single-use packages being arranged against each other with their respective lying planes respectively parallel to form a stack; a pusher operable in the containment chamber against said stack so that a terminal single-use package of said stack is retained against a bottom wall of said chamber; an extractor movable between a waiting position in which it engages the terminal single-use package within the containment chamber and an extracted position in which the terminal single-use package is extracted through an outlet slot arranged in proximity to the bottom wall to expose the respective condom outside the box-shaped casing in a condition suitable for wearing.

In a further aspect, a single-use package of condoms, to be used preferably in conjunction with said case is an object of the invention, comprising:
a condom rolled around its own terminal edge extending around an inlet opening according to a substantially flat configuration; a support frame circumscribing the condom substantially in a lying plane coplanar with the terminal edge of the condom itself, wherein the support frame comprising a mutually separable front portion and rear portion, which rotatably engage the terminal edge in correspondence with respective mutually opposite zones along the perimeter development thereof; a protective film that surrounds the condom and at least a part of the support frame; wherein the protective film is frangible upon mutual separation of the front portion and the rear portion along an opening direction parallel to the lying plane, to make the inlet opening of the condom accessible along a direction normal to said lying plane.

In at least one of the aforesaid aspects, a convenient embodiment may also include at least one of the following preferred features.

Conveniently, the containment chamber is delimited between said bottom wall and an opposite top wall, a pair of respectively opposite frontal walls, and a pair of respectively opposite side walls.

Conveniently, between the waiting position and the extracted position, the extractor makes a stroke that is longer than a width of the containment chamber, which can be detected between the frontal walls.

Conveniently, said outlet slot is obtained in one of the frontal walls.

Conveniently, the extractor has a pair of coupling pins operatively engageable with respective thrust shoulders borne by each of the single-use packages.

Conveniently, the coupling pins are slidably engaged through guide slots arranged in the side walls.

Conveniently, the extractor comprises a pair of thrust arms arranged outside the containment chamber and each bearing one of said coupling pins.

Conveniently, each thrust arm extends adjacent to one of the side walls.

Conveniently, the thrust arms are mutually interconnected by a connecting crossbar.

Conveniently, the extractor also comprises a control lever oscillatably constrained with respect to the box-shaped casing and engaging the thrust arms.

Conveniently, the control lever is oscillatably constrained to the box-shaped casing according to a hinging-axis X arranged in correspondence to the top wall.

Conveniently, the control lever has two mutually parallel opposite walls delimiting at least one guide cavity.

Conveniently, the connecting crossbar is slidably engaged through the guide cavity.

Conveniently, the control lever is movable between a rest position wherein it is parallelly approached to the box-shaped casing, and a trigger position wherein it diverges angularly from the box-shaped casing.

Conveniently, the thrust arms are movable with respect to the guide cavity between a rest position at which their distal ends opposite to the coupling pins and/or the connecting crossbar are close to the hinging axis of the control lever, and a trigger position at which said distal ends are moved away from the hinging axis.

Conveniently, in the rest position, the distal ends and/or the connecting crossbar are arranged at a retaining recess provided in the guide cavity.

Conveniently, in the trigger position, the distal ends a and/or the connecting crossbar are moved away from the hinging axis beyond a geometric plane normal to the longitudinal extension of the guide cavity and containing the coupling pins.

Conveniently, in the trigger position, the distal ends and/or the connecting crossbar are positioned against at least one stop seat arranged along the guide cavity, preferably at the end of the control lever.

Conveniently, the guide cavity has a progressively decreasing width as it moves away from the hinging axis.

Conveniently, wherein the sliding of the distal ends and/or of the connecting crossbar towards the stop seat determines an elastic split of the guide cavity.

Conveniently, in the trigger position, the thrust arms are oriented parallel to the lying plane of the terminal single-use package, preferably at or near it.

Conveniently, lateral shoulders projecting externally from the containment chamber to provide respective resting seats for the thrust arms in the trigger condition are further provided.

Conveniently, at least one of said frontal walls has an openable access door to enable access to the containment chamber.

Conveniently, the pusher comprises a spring and a plate pushed elastically by the spring towards the bottom wall of the containment chamber.

Conveniently, at least one of the front and rear portions of the support frame, preferably the front portion, has at least one thrust shoulder facing the rear portion along the opening direction.

Conveniently, at least one of the front and rear portions of the support frame, preferably the front portion, has a primary branch extending transverse to the opening direction, and a pair of connecting branches extending from the respectively opposite ends of the primary branch to the other one of said front and rear portions.

Conveniently, the ends of the connecting branches that are opposite to the primary branch act against the other one of said front and rear portions to keep the front portion and the rear portion mutually approached in a compacted condition.

Conveniently, in the compacted condition the support frame gives the terminal edge of the condom a perimeter development elongated transversely to the opening direction.

Conveniently, the ends of the connecting branches are joined in one piece to the rear portion, at in correspondence with frangible areas having a small thickness.

Conveniently, at least one thrust shoulder is defined along one of the connecting branches of the front portion.

Conveniently, at least two of these thrust shoulders are provided, each one borne by one of the connecting branches.

Conveniently, said at least one thrust shoulder is defined within a through seat obtained across the respective connecting branch.

Conveniently, the through seat has a substantially L-shaped configuration, with a constraint section extending parallel to the opening direction to define an undercut bearing the thrust shoulder, and an access side extending perpendicular to the opening direction and opening at an edge of the connecting branch.

Conveniently, said at least one thrust shoulder may be engaged by the extractor, which is movable along the opening direction to determine the front portion moving away from the rear portion.

Conveniently, at least one of said front and rear portions, preferably the rear portion, has at least one abutment shoulder projecting transversely to the opening direction and facing the direction opposite to the thrust shoulder.

More in particular, the abutment shoulder faces a direction opposite to the direction which the thrust shoulder faces.

Conveniently, the abutment shoulder projects from the lying plane.

Conveniently, said at least one thrust shoulder is arranged on the front portion, and said at least one abutment shoulder is arranged on the rear portion.

Conveniently, the abutment shoulder is configured to rest against a stop surface and retain the rear portion while the extractor acts on the front portion.

Conveniently, in the condition of use the terminal edge and/or the inlet opening of the condom has a substantially circular development.

Conveniently, each of the front and back portions engages the terminal edge of the condom in a coupling seat.

Conveniently, each of said coupling seats has, in a plane orthogonal to the lying plane, a profile in a substantially circular transversal section circumscribing the terminal edge externally to the inlet opening, according to an arc of amplitude greater than 180°.

Conveniently, each coupling seat has an enlarged and rounded edge, acting against the outer surface of the condom near the terminal edge.

Conveniently, during the translation towards the extraction position, and/or along the opening direction, the extractor causes a fracture in the protective film to expose the condom to a condition suitable for wearing.

Conveniently, during the translation towards the extracted position, the front portion of the single-use package is moved away from the rear portion.

Conveniently, while advancing towards the extracted position the extractor completes a stroke in which the terminal single-use package is extracted through the outlet slot and a second portion in which the front portion of the support frame is moved away from the rear portion.

Conveniently, between the waiting position and the extracted position, the extractor overcomes an intermediate position where the rear portion of the support frame rests in abutment against a stop surface arranged in the containment chamber to retain the rear portion as the extractor advances towards the extraction position.

Conveniently, the translation movement of the extractor from the waiting position to the extracted position takes place according to a stroke having a first portion wherein the rear portion of the terminal single-use package translates together with the front portion, and a second portion wherein the rear portion is stopped against a stop surface arranged in the containment chamber while the front portion moves away from the rear portion.

Conveniently, the bottom wall has an expulsion slot configured to evacuate the rear portion of the support frame from the containment chamber.

Conveniently, a removable collection container is also provided which may be engaged against the bottom wall, outside the containment chamber.

Further features and advantages will better appear from the detailed description of a preferred, but not exclusive, embodiment of a case for the storage and easy wearing of condoms, according to the present invention. Such description will be set forth herein below with reference to the accompanying drawings, provided for merely indicative and therefore non-limiting purposes, wherein:

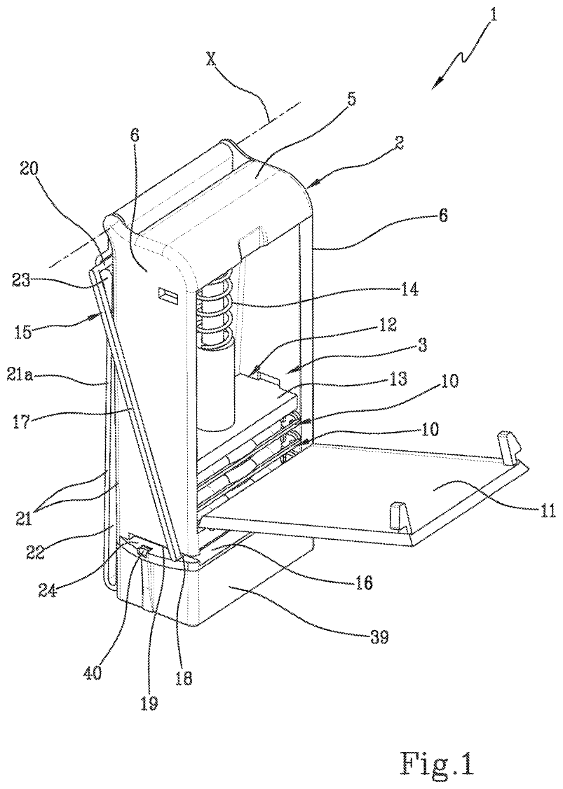

FIG. 1 shows in a perspective view a case for the storage and easy wearing of condoms according to the present invention, with an access door in an opening condition;

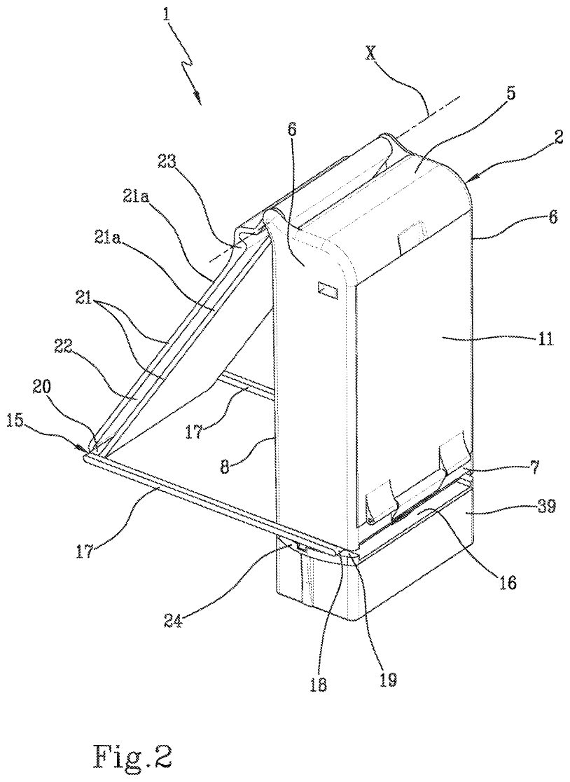

FIG. 2 shows the case of FIG. 1 arranged to be used for dispensing a condom;

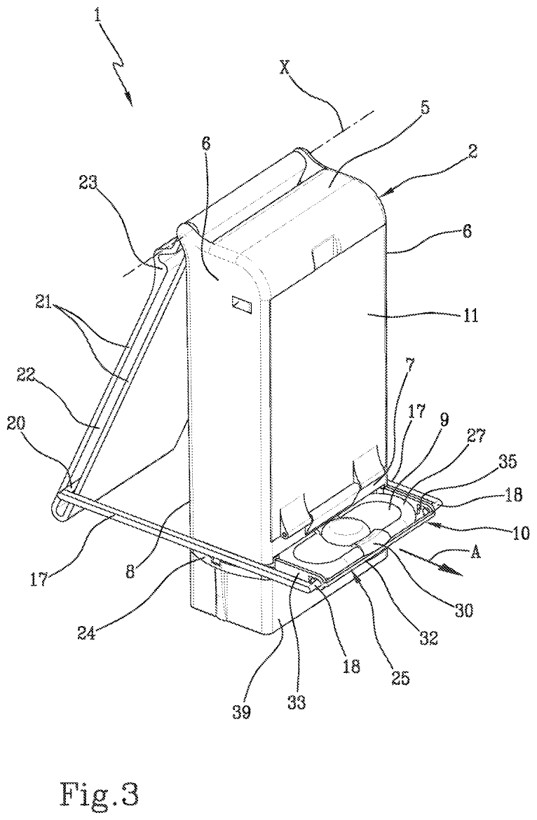

FIG. 3 shows an operating step wherein a condom is about to be extracted from the case;

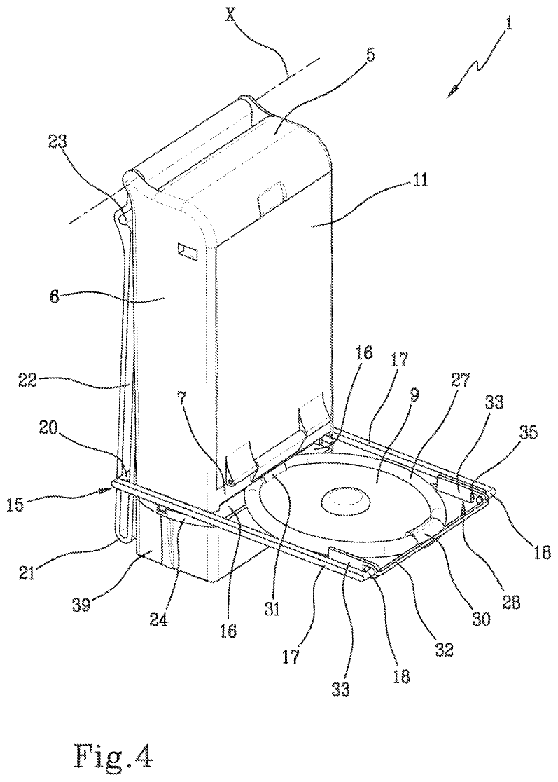

FIG. 4 shows the case after the condom has been extracted, arranged in a wearing condition;

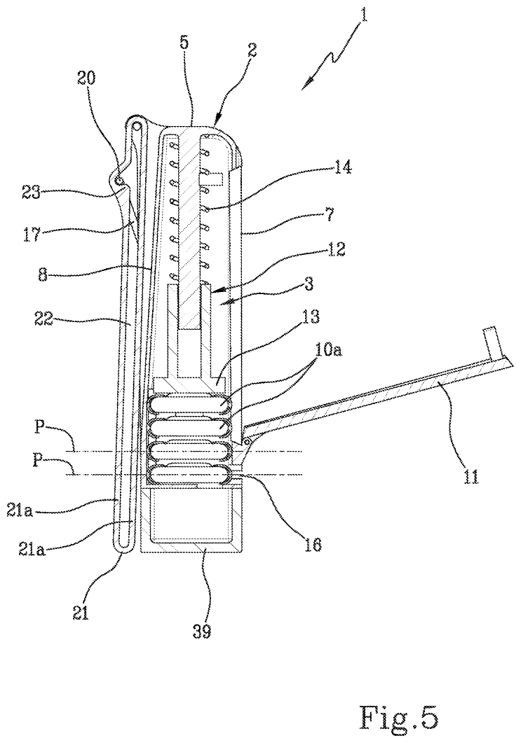

FIGS. 5 to 8 show the case sectioned in a vertical plane, each one in one of the conditions shown in FIGS. 1 to 4;

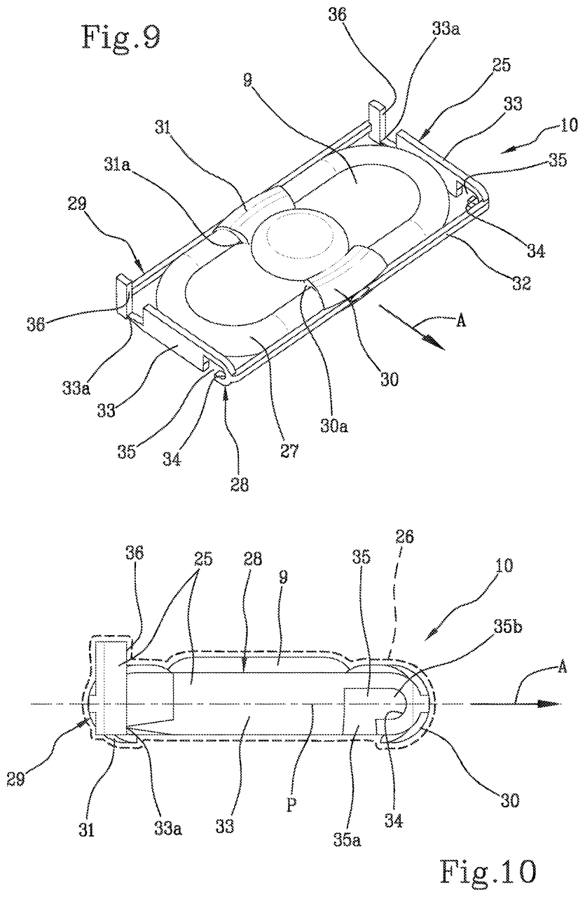

FIG. 9 shows in a perspective view a single-use package that can be used in combination with the case;

FIG. 10 shows the single-use package of FIG. 9 in a side view;

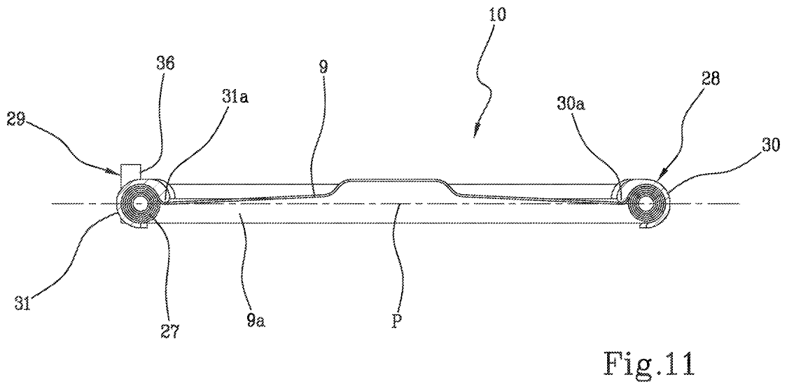

FIG. 11 shows the single-use package in the opened condition sectioned according to a vertical plane.

With reference to the appended figures, a case for the storage and easy wearing of condoms according to the present invention is collectively referred to as 1.

The case 1 comprises a box-shaped casing 2, for example made of plastic and/or metal material, defining therein a containment chamber 3 delimited between said bottom wall 4 and a top wall 5 opposite thereto, a pair of side walls 6 parallelly opposite to each other, and a pair of respectively front 7 and rear 8 frontal walls, also parallelly opposite to each other.

The containment chamber 3 is suitable for housing a plurality of condoms 9, each one arranged in a respective single-use package 10 having a substantially flattened shape in a respective lying plane P. The single-use packages 10 are suitable to be inserted into the containment chamber 3 after opening an access door 11 arranged in the front frontal wall 7 to enable access to the containment chamber itself.

The single-use packages 10 are arranged one against the other with the respective lying planes P respectively parallel and overlapping to each other to form a stack 10a (FIG. 5) which extends moving away from the bottom wall 4.

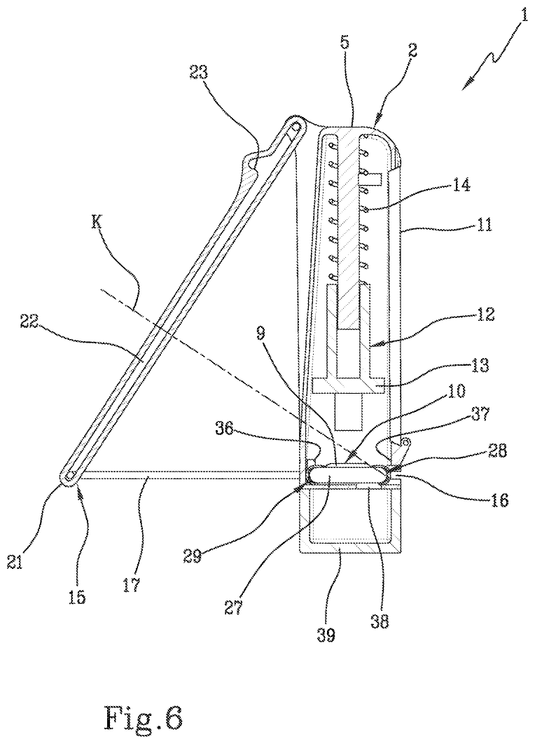

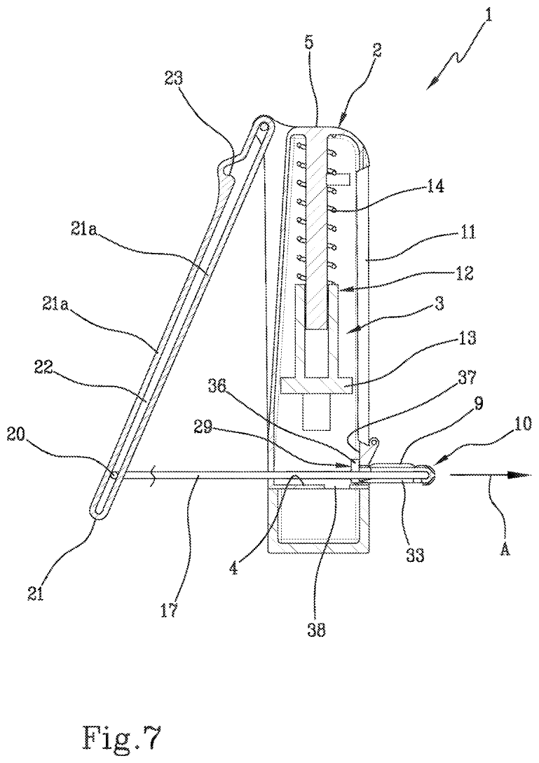

A pusher 12, comprising for instance a plate 13 elastically pushed by the spring 14, operates in the containment chamber 3 against the stack 10a (not illustrated in FIGS. 6 and 7) so that a terminal single-use package 10, located at the base of the stack itself, is constantly pushed against the bottom wall 4.

The case 1 further comprises an extractor 15, movable between a waiting position in which it engages the terminal single-use package 10 within the containment chamber 3, and an extracted position wherein the terminal single-use package 10 is extracted through an outlet slot 16 obtained in the front frontal wall 7, near the bottom wall 4. In the extracted position, the condom 9 borne by the terminal single-use package 10 is fully exposed externally to the box-shaped casing 2, in a condition suitable for wearing.

In the example shown, the extractor 15 comprises a pair of thrust arms 17 arranged outside the containment chamber 3, preferably each adjacent to one of the side walls 6. Each of the thrust arms 17 bears, at one end thereof, a coupling pin 18 slidably engaged through a respective guide slot 19 provided in the side wall 6. The coupling pins 18 may be operatively engaged with the terminal single-use package 10 to determine movement through the outlet slot 16 after actuating the extractor 15.

On the side opposite the coupling pins 18, a connecting crossbar 20 mutually interconnects the thrust arms 17, preferably at the respective distal ends 17a thereof.

The box-shaped casing 2 is oscillatably constrained, preferably according to a hinging axis X arranged near the top wall 5, a control lever 21 movable between a rest position, wherein it is parallelly approached to the box-shaped casing 2 as shown in FIG. 1, and a trigger position, wherein it diverges angularly from the box-shaped casing 2, as for example visible in FIG. 2. One or more springs and/or other elastic return elements (not shown) may possibly be arranged between the control lever 21 and the box-shaped casing 2, such as at the hinging axis X, in order to return the control lever to the rest position.

The control lever 21 may have a plate-shaped structure, defined by two walls 21a parallelly opposite to each other, so as to delimit at least one guide cavity 22, preferably a through-guide, running longitudinally through the control lever 21 itself. The connecting crossbar 20 is slidably engaged through the guide cavity 22, so as to engage the thrust arms 17 to the control lever 21. The sliding engagement of the connecting crossbar 20 along the guide cavity 22 makes the thrust arms 17 be movable with respect to the control lever 21 as it moves between the rest position and the trigger position: in the rest position, the connecting crossbar 20, as well as the distal ends 17a, are brought closer to the hinging axis X of the control lever 21, and preferably retained in a retaining recess 23 arranged in the guide cavity 22. In the trigger position, the connecting crossbar 20 and the distal ends 17a are moved away from the hinging axis X, preferably beyond a geometric plane K normal to the longitudinal development of the guide cavity 22 and containing the coupling pins 18. In the trigger position, the connecting crossbar 20 and the distal ends may be positioned against a stop seat 23 arranged along the guide cavity 22 at the end of the control lever 21. It may be provided that the guide cavity 22 has a width, defined by the distance between the opposite walls 21a, which progressively decreases as it moves away from the hinging axis X, so that the sliding of the connecting crossbar 20 towards the stop seat 23 causes an elastic slip of the opposite walls. The elastic return action of the opposite walls 21a adequately restrains the connecting crossbar 20 in the trigger position.

When the trigger position is reached, the thrust arms 17 are preferably oriented parallel to the lying plane P of the terminal single-use package 10, preferably coinciding with or close thereto. Two or more lateral shoulders 24 projecting externally from the side walls 6 provide a support seat for each of the thrust arms 17 in the trigger position.

As better shown in the FIGS. 1 to 11, in each of the single-use packages 10 the condom 9 is engaged to a support frame 25, and a protective film 26 surrounds the condom 9 and at least part of the support frame itself.

In an manner known in itself, the condom 9 is rolled around its own terminal edge 27 extending in a substantially flat configuration around an inlet opening. The support frame 25 circumscribes the condom 9 substantially in the lying plane P, coplanar to the terminal edge of the condom 9 itself.

The support frame 25 comprises a mutually separable front portion 28 and a rear portion 29, which engage the terminal edge 27 of the condom 9 at respective mutually opposite areas along the perimeter development thereof. More particularly, the engagement of the condom 9 preferably occurs at a front coupling seat 30 and a rear coupling seat 31, borne by the front portion 28 and rear portion 29, respectively. As visible in FIG. 11, each of the coupling seats 30, 31 has, in a plane orthogonal to the lying plane P, a profile in a substantially circular cross-section, which circumscribes the terminal edge 27 of the condom 9 outside the inlet opening 9a, according to an arc preferably having an amplitude greater than 180°, preferably comprised between 210° and 270°. Each of the coupling seats 30, 31 may have an enlarged and rounded edge 30a, 31 a, acting against a surface of the condom 9 outside the inlet opening 9a, close to the terminal edge 27, to facilitate unrolling it.

The protective film 26 may be made of a plastic and/or aluminium material or a multilayer composition of different materials, with a thickness and/or pre-breaking areas such that it is frangible as a result of the front portion 28 and the rear portion 29 moving away from each other along an opening direction A parallel to the lying plane P and the sliding direction of the coupling pins 18 along the respective guide slots 19.

Preferably, the front portion 28 of the support frame 25 has a primary branch 32 extending transverse to the opening direction A, and a pair of connecting branches 33 extending from the respectively opposite ends of the primary branch 32 towards the rear portion 29. The ends of the connecting branches 33 opposite the primary branch 32 act against the rear portion 29 to keep the front portions 28 and rear portions 29 at a close distance from each other, in a compacted condition such as to confer to the terminal edge 27 of the condom 9 an elongated perimeter development transverse to the opening direction A. The ends of the connecting branches 33 may be joined in one piece to the rear portion 29, at frangible areas 33a of a small or reduced thickness.

Along the connecting branches 33, near their ends close to the primary branch 32, respective thrust shoulders 34 facing the rear portion 29 along the opening direction A are defined. The thrust shoulders 34 cooperate with the coupling pins 18 of the extractor 15, to determine the extraction of the terminal single-use package 10 through the outlet slot 16.

Preferably, each thrust shoulder 34 is defined within a through-hole 35 obtained through the respective connecting branch 33 according to a substantially L-shaped configuration, with an access side 35a extending perpendicularly to the opening direction A and opening on an edge of the connecting branch 33 to facilitate the entry of the respective coupling pin 18, and a constraint portion 35b extending parallel to the opening direction A to define an undercut bearing the thrust shoulder 34.

The rear portion 29 has at least one or more abutment shoulders 36 projecting from the lying surface P transversely to the opening direction A, and facing the direction opposite to the direction which the thrust shoulder 34 faces. Each abutment shoulder 36 is configured to abut against a defined stop surface 37 within the containment chamber 3, e.g. close to the outlet slot 16, to retain the rear portion 29 during the action of the extractor 15 on the front portion 28, when the single-use package 10 is extracted from the outlet slot itself.

The use of the case 1 provides that the single-use packages 10 are loaded inside the containment chamber 3 upon opening the access door 11 on the anterior front wall 7.

In the rest condition, the control lever 21 is positioned adjacent to the rear front wall 8 of the box-shaped casing 2 while the thrust arms 17, with their respective coupling pins 18 inserted in the through-holes 35, assume a diagonal orientation with respect to the side walls 6, as shown in FIG. 1. The connecting crossbar 20 is engaged in the retaining recess 23 so as to stabilise the control lever 21 and thrust arms 17 in the rest position. In this circumstance, the case takes up very little space and may be conveniently placed, for example, in a pocket or purse.

For use purposes, the connecting crossbar 20 is manually disengaged from the retaining recess 23 and moved along the guide cavity 22 away from the hinging axis X. The thrust arms 17 consequently rotate about the axes of the coupling pins 18, while the control lever 21 moves away from the rear front wall 8 until it assumes the trigger position shown in FIGS. 2 and 5. When the trigger position is reached, a manual action on the control lever 21 causes it to rotate about the hinging axis X and the consequent sliding of the thrust arms 17 from the waiting position towards the extracted position.

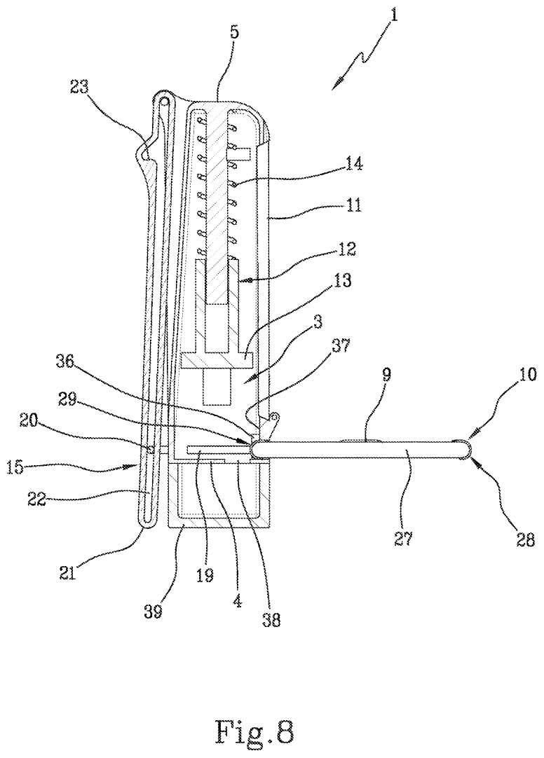

The translation of the extractor 15 from the waiting position to the extracted position occurs according to a stroke having a length greater than the internal width of the containment chamber 3, which may be detected between the front 7 and rear 8 frontal walls. More particularly, the stroke performed by the extractor 15 has a first portion in which the entire terminal single-use package 10 is extracted through the outlet slot 16 until it reaches an intermediate position, shown in FIG. 6, wherein the abutment shoulder 36 borne by the rear portion 29 of the support frame 25 abuts against the stop surface 37. The further rotation of the control lever 21 approaching the box-shaped casing 2 results in the execution of a second stroke portion of the ejector 15, during which the front portion 28 of the support frame 25 moves away from the rear portion 29 while the latter remains stationary against the stop surface 37, until it reaches the extracted position shown in FIGS. 4 and 8. In this circumstance, the thrust exerted by the extractor 15 on the front portion 28 causes it to separate from the rear portion 29, with the simultaneous fracture of the protective film 26 surrounding the terminal single-use package 10 and of any frangible areas 33a.

When the extraction position is reached, the condom 9 is exposed in the condition of use suitable for wearing wherein, as visible in FIG. 4, its terminal edge 27 has a substantially circular perimeter development and the inlet opening 9a, also substantially circular, is accessible along a direction normal to the lying plane P.

In this circumstance, the coupling seats 30, 31 rotatably retain the terminal edge 27 of the condom 9 so that it can easily rotate on itself, to ease unrolling the condom 9 while being worn and subsequently disengaging it from the support frame 25.

An expulsion slot 38 may be provided in the bottom wall 4 to ease the evacuation of the rear portion 29 from the containment chamber 3, and to allow positioning a new single-use package 10 against the bottom wall 4 itself.

When the control lever 21 is released, the thrust arms 17 and the control lever 21 itself may be manually or elastically returned to the waiting position by moving the connecting crossbar 20 along the guide cavity 22. With a slight elastic deformation of the thrust arms 17 and/or a slight lifting of the stack 10a of single-use packages 10, the coupling pins 18 may be easily introduced into their respective pass-through seats 35 through the access portions 35a, to engage the new terminal single-use package 10 positioned against the bottom wall 4.

The fragments of the support frame 25 and of the protective film 26, as well as the used condom 9, may be collected in a collection container 39 removably engageable against the bottom wall 4, outside the containment chamber 3, e.g. by means of coupling elements 40 preferably snap-acting on the lateral shoulders 24.

1. Case for the storage and easy wearing of condoms, including:
a box-shaped casing defining a containment chamber configured to accommodate a plurality of condoms each arranged in a single-use package having a substantially flattened shape in a respective lying plane, the single-use packages being arranged one against the other with their respective lying planes respectively parallel to form a stack;
a pusher operating in the containment chamber against said stack so that a terminal single-use package of said stack is held against a bottom wall of the chamber itself;
an extractor movable between a waiting position in which it engages the terminal single-use package inside the containment chamber and an extracted position in which the terminal single-use package is extracted through an outlet slot arranged in proximity to the bottom wall to expose the respective condom outside the box-shaped casing, in a use condition suitable for wearing.

2. Case according to claim 1, wherein the extractor has a pair of coupling pins operatively engageable with respective thrust shoulders carried by each of the single-use packages.

3. Case according to claim 1, wherein the extractor comprises a pair of thrust arms arranged externally to the containment chamber and each carrying one of said coupling pins.

4. Case according to claim 3, wherein the extractor also comprises a control lever oscillatably constrained with respect to the box-shaped casing and engaging the thrust arms.

5. Case according to claim 4, wherein the control lever has two parallel opposite walls delimiting at least one guide cavity.

6. Case according to claim 4, wherein the control lever is movable between a rest position in which it is parallel to the box-shaped casing, and a trigger position in which it diverges angularly from the box-shaped casing.

7. Case according to claim 5, wherein the thrust arms are movable with respect to the guide cavity between a rest position at which their distal ends opposite the coupling pins are approached to a hinging axis of the control lever, and a trigger position at which said distal ends are moved away from the hinging axis.

8. Case according to claim 7, wherein at the rest position said distal ends are arranged in correspondence with a retaining recess provided in the guide cavity.

9. Case according to claim 7, wherein at the trigger position, the thrust arms are oriented parallel to the lying plane of the terminal single-use package.

10. Case according to claim 7, further comprising lateral shoulders projecting externally from the containment chamber to provide respective resting seats for the thrust arms at the trigger condition.

11. Case according to claim 1, wherein each of said single-use packages comprises:
said condom rolled around its own terminal edge extending around an inlet opening according to a substantially flat configuration;
a support frame circumscribing the condom substantially in the lying plane coplanar with the terminal edge of the condom itself, wherein the support frame comprises a front portion and a rear portion mutually separable, which rotatably engage the terminal edge in correspondence with respective mutually opposite zones along its perimeter development;
a protective film that surrounds the condom and at least a part of the support frame;
wherein the protective film is breakable upon mutual separation of the front portion and the rear portion along an opening direction parallel to the lying plane, to make the inlet opening of the condom accessible along a direction normal to said lying plane.

12. Case according to claim 11, wherein during advancement towards the extracted position the extractor completes a stroke comprising a first portion in which the terminal single-use package is extracted through the outlet slot, and a second portion in which the front portion of the support frame is moved away from the rear portion.

13. Case according to claim 11, wherein the bottom wall has an expulsion slot configured for the evacuation of the rear portion of the support frame from the containment chamber.

14. Single-use package for condoms, comprising:
a condom rolled around its own terminal edge extending around an inlet opening according to a substantially flat configuration;
a support frame circumscribing the condom substantially in a lying plane coplanar with the terminal edge of the condom itself, wherein the support frame comprises a front portion and a rear portion mutually separable, which rotatably engage the terminal edge in correspondence with respective mutually opposite zones along its perimeter development;
a protective film that surrounds the condom and at least a part of the support frame;
wherein the protective film is breakable upon mutual separation of the front portion and the rear portion along an opening direction parallel to the lying plane, to make the inlet opening of the condom accessible along a direction normal to said lying plane, wherein at least one among the front and rear portions of the support frame has at least one thrust shoulder facing the other portion along the opening direction, while the other of said front and rear portions has at least one abutment shoulder projecting transversally to the opening direction and facing opposite to the thrust shoulder.

15. Package according to claim 14, wherein the abutment shoulder protrudes with respect to the lying plane.

16. Package according to claim 14, wherein at least one of the front and rear portions of the support frame has a primary branch extending transversely to the opening direction, and a pair of connecting branches extending from the respectively opposite ends of the primary branch towards the other of said front portion and rear portion.

17. Package according to claim 16, wherein ends of the connecting branches opposite the primary branch act against the other of said front portion and rear portion to maintain the front portion and the rear portion mutually close together in a compacted condition.

18. Package according to claim 17, wherein in the compacted condition the support frame confers to the terminal edge of the condom a perimetral development elongated transversely to the opening direction.

19. Package according to claim 16, wherein the ends of the connecting branches are joined in one piece to the rear portion, in correspondence with frangible areas having a reduced thickness.

20. Package according to claim 16, wherein said at least one thrust shoulder is defined along one of the connecting branches of the front portion.

21. Package according to claim 16, wherein said at least one thrust shoulder is defined inside a through seat obtained across the respective connecting branch.

22. Package according to claim 21, wherein the through seat has a substantially L-shaped configuration, with a constraint portion extending parallel to the opening direction to define an undercut bearing the thrust shoulder, and an access side extending perpendicular to the opening direction and opening at an edge of the connecting branch.

23. Package according to claim 14, wherein each of the front and rear portions engages the terminal edge of the condom in a respective coupling seat having in a plane orthogonal to the lying plane, a substantially circular cross-sectional profile circumscribing the terminal edge externally to the inlet opening, according to an arc with an amplitude greater than 180°.

24. Package according to claim 23, wherein each coupling seat has an enlarged rounded edge, acting against the external surface of the condom close to the terminal edge.
